SEQUENCE LISTING (2 sequences)

SEQ ID NO: 1 - DNA, 1621 bp, Mycoplasma fermentans
aaggagattatatgaaaaagtcaaaaaaaattttattaggattgagtcctattgctgctattcttcctgcagtagctgtttcttgtggaaacaacgatgaatccaatatttcattcaaagagaaagatattagtaaatataccacaacaaatgctaatggaaaacaagttgttaaaaacgctgaattgttaaaattgaaaccagttcttattacagatgaaggtaaaattgatgataaatcatttaaccaatcagcttttgaagctttaaaagctataaataaacaaactggtattgaaattaacaatgttgaacctagctcaaactttgaaagtgcttacaacagtgcactttcagccggacacaaaatttgagtacttaatggcttcaaacaccaacaatctattaaacaatacattgatgctcacagagaagaacttgaaagaaatcaaatcaaaatcattggtatcgactttgatattgaaacagagtacaagtgattctactcattacaattcaatattaaagaatctgcatttacaacaggctatgcaattgcaagttgattaagtgaacaagatgaaagtaaaagagttgttgcatcatttggtggaggtgcattcccaggtgttacaacatttaacgaaggttttgcaaaaggtattctatactacaaccaaaaacataaatcaagtaaaatttaccacacatcacctgttaaattagactcaggttttactgctggtgaaaaaatgaacactgttattaataatgttttatcttcaacaccagctgatgttaaatacaacccacatgttatcttatctgttgctggacctgctacatttgaaactgtaagattagcaaacaaaggtcaatatgtaattggtgttgactcagaccaaggcatgattcaagacaaagacagaattcttacatcagttctaaaacacattaaacaagctgtttatgaaacattattagatcttattcttgaaaaagaagaaggatataaaccatatgtagttaaagacaaaaaagcagacaaaaaatgaagccactttggaactcaaaaagaaaaatgaatcggtgtcgcagaaaaccacttctcaaatacagaagaacaagcaaaaattaataacaaaattaaagaagcaattaaaatgtttaaagaattaccagaagatttcgttaaatatattaatagtgacaaagctttaaaagatggtaataaaattgacaatgttagtgaaagattagaagcaattatttctgctattaacaaggcagcaaaataattaatcaaaaaaatgctggaaaatatccagcattttttattttaaatatgaaaaaagtatatttttttgttaatttttgaagaaattagataaaacagtttttccgtttttgtcttcaaataagataaataagagaaaaaaggttgtaaaactgcctaagaaaaaaagagttggaaaacataaaagttaaagaccaaattatttaactatttatgactttgagatggatacggttcatgggtgtagaatggatgaatatttcattttgattttaataaatagattgactagagaagtacactttaaaaaaaaaaaaaaaaaaa

SEQ ID NO: 2 - protein, 428 aa, Mycoplasma fermentans
MKKSKKILLGLSPIAAILPAVAVSCGNNDESNISFKEKDISKYTTTNANGKQVVKNAELLKLKPVLITDEGKIDDKSFNQSAFEALKAINKQTGIEINNVEPSSNFESAYNSALSAGHKIWVLNGFKHQQSIKQYIDAHREELERNQIKIIGIDFDIETEYKWFYSLQFNIKESAFTTGYAIASWLSEQDESKRVVASFGGGAFPGVTTFNEGFAKGILYYNQKHKSSKIYHTSPVKLDSGFTAGEKMNTVINNVLSSTPADVKYNPHVILSVAGPATFETVRLANKGQYVIGVDSDQGMIQDKDRILTSVLKHIKQAVYETLLDLILEKEEGYKPYVVKDKKADKKWSHFGTQKEKWIGVAENHFSNTEEQAKINNKIKEAIKMFKELPEDFVKYINSDKALKDGNKIDNVSERLEAIISAINKAAK

(19) United States
(12) Patent Application Publication
(10) Pub. No.: US 2005/0096272 A1
(43) Pub. Date: May 5, 2005

(54) Therapeutic method for solid tumors using M161Ag

(76) Inventors: Tsukasa Seya, Nara-shi (JP); Misako Matsumoto, Ikoma-shi (JP)

Correspondence Address: EDWARDS & ANGELL, LLP, P.O. BOX 55874, BOSTON, MA, 02205, US

(21) Appl. No.: 10/913,053

(22) Filed: Aug. 5, 2004

Related U.S. Application Data
(63) Continuation-in-part of application No. 09/601,371, filed on Dec. 5, 2000, now abandoned.
Application No. 09/601,371 is a continuation of application No. PCT/JP99/00414, filed on Feb. 1, 1999.

(30) Foreign Application Priority Data
Jan. 30, 1998 (JP) 10-32384/1998

(51) Int. Cl. A61K 38/17; A61K 48/00

(52) U.S. Cl. 514/12; 514/44

(57) ABSTRACT

The present invention is to provide cytokine inducers comprising a membrane protein M161Ag which is contained in cells latently infected with Mycoplasma fermentans such as a human myelocytic leukemia cell line P39(+). The present invention relates to cytokine inducers comprising the protein M161Ag or gene recombinant products thereof which are useful for immunomodulators, or therapeutic agents for various immune related diseases and solid tumors.

### TECHNICAL FIELD

This invention relates to cytokine inducers, immunomodulators and immunotherapeutic agents comprising the protein M161Ag, and relates to therapeutic method for solid tumors using the protein M161Ag.

### BACKGROUND ARTS

M161Ag is a membrane protein which is contained in cells latently infected with Mycoplasma fermentans such as a human myelocytic leukemia cell line P39(+), and has functions such as activation of the alternative pathway and adsorption of the complement C3. Isolation and purification of this protein, and preparation of monoclonal antibody have already been reported [Matsumoto et al., J. Exp. Med. 181, 115-125(1995)]. Also, the primary structure has almost been reported and it has been suggested that M161Ag and cDNA encoding the same are possibly be the potent therapeutic agents for such as leukemia because said protein enhances the clearance of the human myelocytic leukemia cells. [Nature Med., 3:1266-1270(1997)] (Japanese Patent Unexamined Publication No.157295/1997).

Mycoplasma fermentans (M. fermentans), is an intracellular parasitic bacterium, which appears to positive under the disease states of immunosuppressive conditions, for example, HIV infected patients, patients with cancer such as leukemia and myeloma and patients with aplastic anemia. Main parasitic host is identified in vitro as human tumor cell strain. It is known that in a positive case of M. fermentans, M161Ag is essentially positive.

In the report by the another group, Mycoplasma is a cause of lymphopenia and is a cofactor of AIDS crisis. Further, M. fermentans has been suggested to induce cytokines as a result of stimlulation of leukocytes system in vitro. As those phenomena can be observed in only limited species including fermentas, it has been considered that some gene products specific to M. fermentas instruct lymphopenia and cytokine induction. However, it was unknown what substance may be responsible for those actions. The present inventors have identified a substance involved in those biological activities of M. fermentas by an assay using purified authentic sample, demonstrated the range of the biological activities, and thereby completed the present invention.

An object of the present invention is to provide cytokine inducers, immunomodulators and immunotherapeutic agents comprising M161Ag and to make good use thereof in treatment for various immunological diseases and solid tumors by applying physiological activity and cytokine inducing activity of M161Ag.

### DISCLOSURE OF INVENTION

The present inventors have succeeded, using purified authentic sample of M161Ag, to induce effectively the inflammatory cytokines such as IL-1β, TNF-α and IL-6 and the lymphocyte-activating cytokines such as IL-10 and IL-12 by stimulating immune competent cells such as monocytes and lymphocytes, and to remove infected cells as a result of apoptosis. Further, it has been revealed through the investigation of the present inventors that protein M161A regresses solid tumors transplanted into mammals, and mammalian spleen cells having solid tumors, whereto administered the protein M161Ag, induces cytotoxic T lymphocytes (CTL) stimulated by solid tumor cells. Consequently, the protein M161A is found to be most likely to regress solid tumors by the co-function of the cytokines and the CTL induced by lymphocyte-activating cytokines. Based on the above findings, the present inventors have completed the invention.

The present invention relates to cytokine inducers, immunomodulators, or therapeutic agents for immune related diseases and solid tumors comprising the protein M161Ag or gene recombinant products thereof.

In addition, the present invention relates to therapeutic method for diseases caused by cytokine deficiency, immune related disease, immunological disease and solid tumors comprising administering therapeutically sufficient amount (effective amount), for example, 10-100 ng/head ( note: the average mouse weighs 20 g) of the protein M161Ag or gene recombinant products thereof to the mammals having disease or solid tumors. Further, the present invention relates to use of the protein M161Ag or gene recombinant products thereof for production of the cytokine inducers, immunomodulators, or therapeutic agents for immune related diseases and solid tumors.

DNA of M161Ag of the present invention can be obtained as genomic DNA from human myelocytic leukemia cell line P39 and human fibroblast cell line W138, in which M. fermentans is infected and proliferated. Since such DNA contains 5 TGA codons (termination codon in E. coli and human; and tryptophan in Mycoplasma), those which are replaced by TGG, consequently the resultant sequence is inserted into the expression vector of E. coli having His-tag such as pET, and then forcibly expressed in cells. Thus obtained large amount of gene recombinant products are purified by nickel Sepharose column followed by MK53 (anti-M161Ag antibody) Sepharose column. The obtained sample is confirmed as E. coli derived pyrogen-free.

Thus purified M161Ag has 45kDa of molecular weight and is bound with lipid such as palmitic acid in N-terminal cysteine. When M161Ag is mixed with cells, a part of M161Ag is incorporated into cells by an action of this lipid. When M161Ag is mixed with peripheral blood, monocytes and monocyte system cells in vitro, the inflammatory cytokines derived from macrophage such as IL-1β, TNF-α and IL-6, and cytokines having modulator activity of lymphocytes such as IL-10 and IL-12 can be identified in the cultured supernatant solution within 24 hours. From comparison of an activity of M161Ag with that of lipopolysaccharide (LPS), in case of using identical weight (such as 10 ng) of these substances, all of the above cytokines are found to be more potentially induced by M161Ag than by LPS. Monocytes used in this experiment are prepared by the method of Karp et al. using elutriation system (Beckman) and have purification of 95% or more. Consequently, it can be concluded that those cytokines were induced by direct stimulation of M161Ag to monocytes.

It can be proved that M161Ag is a bioactive substance, which potentially induces human cytokines, derived from Mycoplasma. In addition, it has been elucidated that M161Ag activated human fundamental immune system (innate immunity) through this action and improved host immunity in the immunosuppressive condition, further resulted the infected cells such as lymphocytes to apoptotic death, and suppressed disease state caused by excess immune activation such as allergy.

As explained hereinabove, it is proved that M161Ag induces cytokines as a modulator in various immune systems through the monocyte system.

Further, the protein M161Ag regressed solid tumors implanted to the mammals and the mammalian spleen cells having solid tumors, whereto administered the protein M161Ag, induced cytotoxic T lymphocytes (CTL) stimulated by solid tumor cells. Through the above observation, the CTL induced by lymphocyte-activating cytokines is most likely to regress the growth of solid tumors by co-function with the cytokines.

Synthesis of DNA sequence of the above described protein M161Ag comprising substitution in DNA codons, e.g. substitution from TGA to TGG, was carried out in E. coli. A large amount of recombinant DNA products were purified through nickel Sepharose column followed by MK53 (anti-M161Ag antibody) Sepharose column by the same method as above and obtained recombinant gene product having 5 amino acid substitutions in the amino acid sequence of M161Ag. Resultant products were examined by the same method as above and revealed to have the same activity as the protein M161Ag.

### BRIEF DESCRIPTION OF DRAWING

FIG. 1 indicates inducing action of the protein M161Ag of the present invention on IL-1β, TNF-α and IL-6 in the monocyte cell THP-1. In FIG. 1, the left graph shows data from cell lysates and the right graph shows data in the conditioned medium.

FIG. 2 indicates inducing action of the protein M161Ag of the present invention on IL-1β, TNF-α and IL-6 in the purified blood monocytes. In FIG. 2, the left graph shows data from cell lysates and the right graph shows data in the conditioned medium.

FIG. 3 indicates inducing action of the protein M161Ag of the present invention on IL-10 (FIG. 3, left) and IL-12 (FIG. 3, right).

### BEST MODE OF CARRYING OUT THE INVENTION

The cytokines induced by the protein M161Ag of the present invention are known to have various bioactivities. For example, IL-1 is known to have activities of T cell activation, neutrophil activation, stimulation of antitumor activity, proliferation of fibroblasts and increase of ACTH and GH. TNF-α is a factor involved in proliferation and differentiation of cells, and especially is a factor having tumor necrotizing action. It is also reported to increase production of prostaglandins and platelet activating factors, more over to have antiviral activity.

IL-6 has functions such as to stimulate differentiation of B cells to antibody production cells as well as to stimulate differentiation and proliferation of T cell and macrophage. IL-10 is a factor having an action to proliferate T cell. IL-12 is a factor having actions to activate cytotoxic T cell (CTL) and NK cell.

M161Ag, in its cytokine inducing activity, can be applied for treatment of immune related diseases and cancers.

Since the active component of the present invention is to activate human innate immunity system, it is useful for therapeutic agents for immune related diseases involved in the innate immunity system and also for solid tumors. Examples of immunological diseases include such as allergic diseases and autoimmune diseases. Examples of solid tumor include mammalian, particularly human carcinomas, such as melanoma, pulmonary carcinoma (excluding small cell lung carcinoma), hepatic carcinoma, renal carcinoma, prostrate carcinoma, gastoric carcinoma, colon carcinoma, breast carcinoma, carcinoma uteri and glioblastoma.

The protein M161Ag of the present invention can be the extract of cells such as cell line P39(+), and can also be the expression product (gene recombinant product) by using DNA with various host cells as disclosed in Japanese Patent Unexamined Publication No.157295/1997.

The protein M161Ag of the present invention, wherein one or more amino acids may be deleted, one or more amino acids may be substituted to other amino acids, or one or more amino acids may be added as far as its activity is maintained. Generally, thus modified protein is not particularly limited in the identity with the protein M161Ag as far as the cytokine inducing property of the protein M161Ag is maintained, however, it is preferable to have 50 % identity or more, more preferably 70% or more, still more preferably 90% or more.

M161Ag of the present invention may be used as it is in the form of protein, however it may be used in the form modified (acylation or so) with fatty acid having 12 to 20 carbon atoms such as palmitic acid in the N-terminal or at the suitable position in the amino acid residues, or may be modified so as to enhance the selectivity targeting the tumor cells by binding with the antibody specific to cells such as tumor cells.

The administration amount of the protein M161Ag of the present invention as an active ingredient, differs depending on conditions of patients and types of diseases though, is generally 500 ng-5 μg/kg/day and is administered once a week.

Intradermal injection is preferable for an administration route, but is not particularly limited to as far as it is parenteral such as rectal administration.

Formulation for administration is not particularly limited but can be used the conventional formulation for protein. Emulsion such as oil-in-water emulsion is more preferable. Concentration of the protein M161Ag in pharmaceutical composition is not particularly limited but preferably is from about 1-50% (by weight), more preferably from about 10-20%. Emulsion such as liposome preparation can also be used.

### EXAMPLES

The present invention is explained more concretely by the following examples, but is not construed as limiting by these examples.

### Example 1

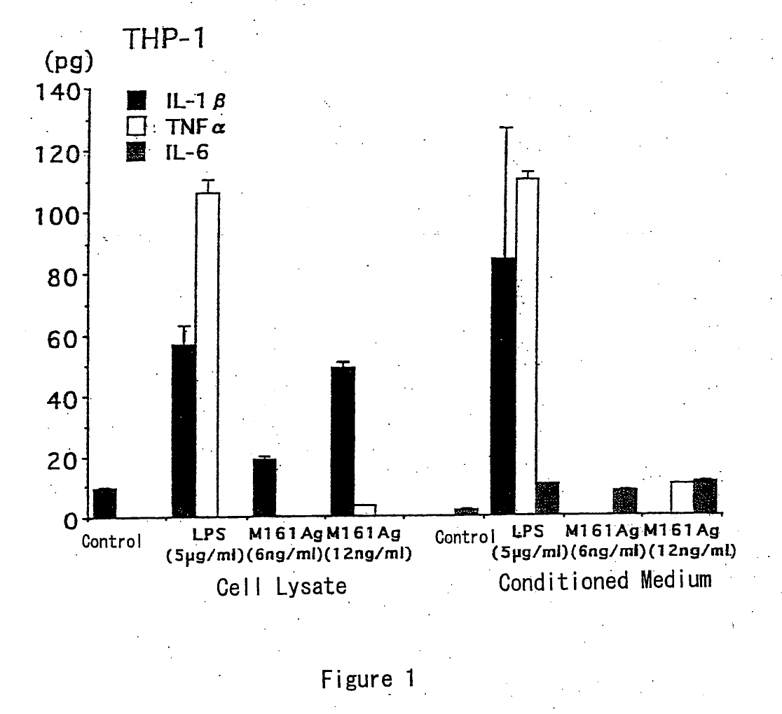

M161Ag, 6 ng and 12 ng, respectively, was added to the monocyte cell system line THP-1 (10⁶). Culture supernatant was collected after 24 hours and various cytokines were quantitatively assayed by sandwich ELISA. FIG. 1 shows results of assay as well as comparative data of using LPS.

### Example 2

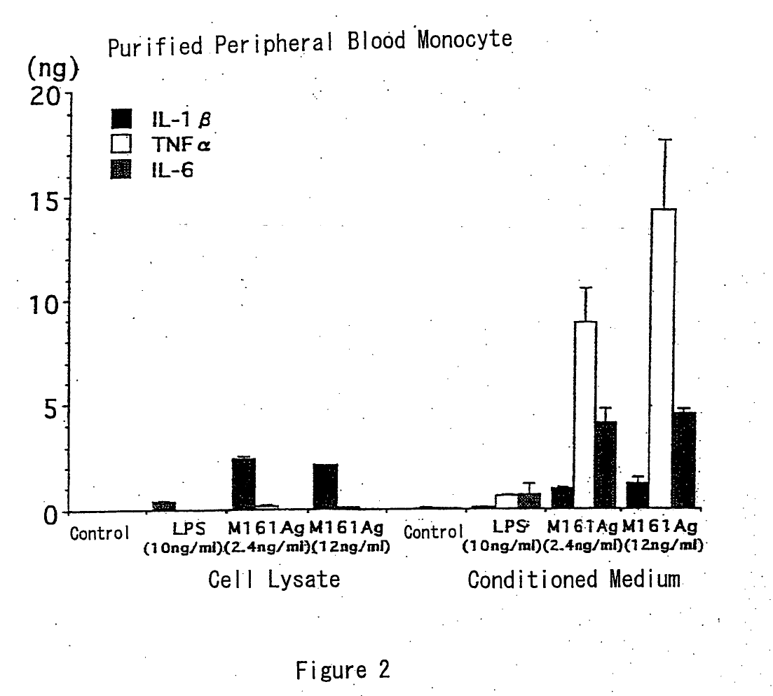

M161Ag, 2.4 ng and 12 ng, respectively, was added to the purified peripheral blood monocytes (10⁶). Culture supernatant was collected after 24 hours and various cytokines were quantitatively assayed by sandwich ELISA. FIG. 2 shows results of assay as well as comparative data of using LPS.

### Example 3

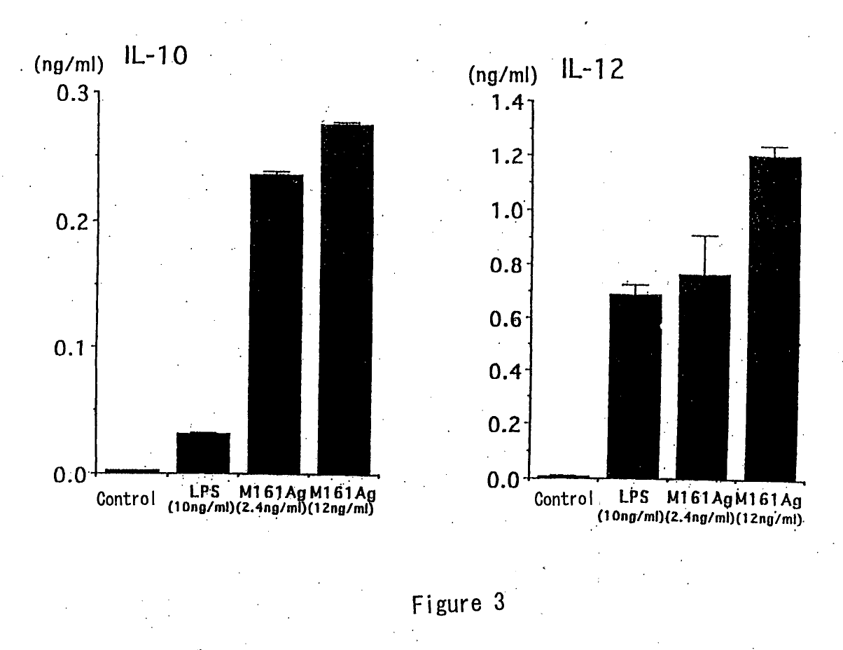

M161Ag, 2.4 ng and 12 ng, respectively, was added to the purified peripheral blood monocytes (10⁶). After 24 hours, IL-10 and IL-12 were quantitatively assayed. Results are shown in FIG. 3.

### Example 4

B16 melanoma (5×10⁵ cell) were dorsally implanted into C57BL6mouse (6 mice, weighs 18 g -25 g) (average weighs 20 g) and tumor diameter was measured after 2 weeks (at Day14). Then mice were divided into 3 test groups (2 mice/group) and the first therapy was conducted by administering BCG-CWS (cell wall skeleton of mycobacterium bovis bacillus calmette-guerin) or M161Ag. The second therapy was conducted after 3 weeks (at Day21) and third therapy was conducted after 4 weeks (at Day 28). Tumor volume ( 0.4×major axis×(minor axis)²) was measured before each therapy and at 5 week (at Day35). Results are shown in Table 1.

Administration procedures are as follows:

M161Ag administration group: M161Ag is suspended in emulsion buffer* with concentration of 10% and administered 50 μg/head by intradermal injection.

BCG-CWS administration group: BCG-CWS is suspended in emulsion buffer* with concentration of 10% and administered 20 μg/head by intradermal injection.

TABLE 1
Average tumor volume
(S.E.)

Weeks

| | 2 | 3 | 4 | 5 |
|---|---|---|---|---|
| Control | 0.25 | 1.58 | 5.43 | 17.11 |
| | (0.12) | (0.82) | (2.54) | (5.87) |
| M161Ag + B16 | 0.17 | 0.49 | 1.72 | 5.76 |
| | (0.11) | (0.19) | (0.96) | (2.70) |
| BCG-CWS + B16 | 0.20 | 0.87 | 3.58 | 8.59 |
| | (0.13) | (0.31) | (1.28) | (3.27) |

The results show that M161Ag has the same or more tumor regression function than BCG-CWS known as anti-tumor agent.

### Example 5

After measuring the tumor diameter at 5 week in Example 4, spleen cell of mice were collected. After in vitro stimulation (5 days) on melanoma cell line B16, cytotoxic activity (% killing) at each E/T ratio (E: effector (lymphocyte), T: target (tumor cell)) was determined by 51Cr release assay.

In vitro stimulation was conducted by the procedures as follows:

Tumor cell lysate was added to the mouse spleen cell as antigen and stimulated 5 days. The collected spleen cell, as an effector cell, was mixed with tumor cell which has been pre-labeled with chromium (51Cr).

51Cr release assay: Cytotoxic assay for targeting (tumor) cells wherein CTL was used for effector cell. (refer to Akazawa et al., Cancer Research, vol.64, p757-764, 2004)

Results were shown in Table 2.

TABLE 2
Average tumor volume
(S.E.)

| | E/T ratio | | |
|---|---|---|---|
| | 3 | 10 | 30 |
| Control | 1.11 | −1.33 | −0.22 |
| | (0.42) | (3.10) | (1.04) |
| M161Ag + B16 | 1.38 | 7.86 | 18.48 |
| | (0.97) | (1.13) | (1.78) |
| BCG-CWS + B16 | 2.80 | 8.62 | 19.32 |
| | (1.60) | (1.05) | (1.08) |

Industrial Applicability

The present invention provides pharmaceutical compositions comprising novel bioactivity of M161Ag, membrane protein of Mycoplasma fermentans. M161Ag shows potent cytokine inducing activities and can be used as immunomodulators for allergic and immunosuppressive conditions, and therapeutic agent for solid tumors.

1. A therapeutic method for solid tumors of mammals comprising administering therapeutically effective amount of the protein M161Ag or gene recombinant products thereof.

2. The therapeutic method according to claim 1, wherein solid tumors are carcinomas.

3. The therapeutic method according to claim 1 or 2, wherein solid tumors are melanomas.

4. The therapeutic method according to claim 1 or 2, wherein the protein M161Ag is a polypeptide having an amino acid sequence as set forth in SEQ ID NO:2.

5. The therapeutic method according to claim 1 or 2, wherein the protein M161Ag is modified with fatty acid in the N-terminal.

6. The therapeutic method according to claim 1 or 2, wherein the recombinant gene product is a polypeptide having substantially the same activity as the protein M161Ag, and having an amino acid sequence wherein one or more amino acids are deleted, substituted or added so as to exhibit 50% or more identity with amino acid sequence as set forth in SEQ ID NO:2.
